(19) United States
(12) Patent Application Publication
(10) Pub. No.: US 2012/0164717 A1
(43) Pub. Date: Jun. 28, 2012

(54) Identity profiling of cell surface markers

(76) Inventor: Joseph Irudayaraj, West Lafayette, IN (US)

(21) Appl. No.: 12/218,129

(22) Filed: Jul. 11, 2008

Related U.S. Application Data
(60) Provisional application No. 60/959,947, filed on Jul. 18, 2007.
(60) Provisional application No. 61/009,988, filed on Jan. 3, 2008.

(51) Int. Cl. C12M 1/34

(52) U.S. Cl. 435/287.2; 435/288.7

(57) ABSTRACT

An apparatus and method of characterizing cells having surface markers includes the use of functionalized probes, the probes having absorption spectra characteristic of a probe geometry. The probes are functionalized with biomolecules (biomarkers) capable of binding to the surface markers, and cells having a particular combination of probes having specific bound biomarkers are measured using a spectrum analyzer. A plurality of surface markers may be simultaneously measured using the spectral properties of the probes to differentiate the cells. The relative abundance of a plurality of surface markers may be determined simultaneously. Functionalized probes with several different aspect ratios were used in an experiment to demonstrate the use of functionalized probes for characterizing cancer cells. The functionalized gold nanorod probes when attached to magnetic particles may be used as a dual contrast agent for magnetic resonance imaging (MRI) and optical imaging and as a targeted drug delivery system.

This application claims the benefit of U.S. provisional application Ser. No. 60/959,947, filed on Jul. 18, 2007 and U.S. provisional application Ser. No. 61/009,988, filed on Jan. 3, 2008, each of which are incorporated herein by reference.

### TECHNICAL FIELD

The present application relates to an apparatus and method of using nanomaterials for determining the populations of cells having specific biomarkers.

### BACKGROUND

The development of biosensors for detection, monitoring and characterization of a variety of molecular interactions are important for disease diagnosis, drug discovery, proteomics, and detection of biological warfare agents. Fundamentally, a biosensor is constructed by coupling a ligand to its receptor complement via an appropriate signal transduction element. Various signal transduction mechanisms have been explored as biosensing schemes, including optical, radioactive, electrochemical, piezoelectric, magnetic, micromechanical, IR and Raman spectroscopy, and mass spectrometry.

Flow cytometry is a technique for counting, examining, and sorting microscopic particles suspended in a stream of fluid. It allows simultaneous multi-parametric analysis of the physical and/or chemical characteristics of single cells.

The technology has applications in a number of fields, including molecular biology, pathology, immunology, plant biology and marine biology. In the field of molecular biology it is especially useful when used with fluorescently tagged antibodies. These specific antibodies bind to antigens (surface markers) on the target cells.

A flow cytometry system may comprise a flow cell in which a liquid stream (sheath fluid) carries and aligns the cells so that they pass through the light beam for sensing. A light source for exciting the fluorescent markers may be, for example, a lamp (such as a mercury or xenon source) or one or more lasers. The lasers may range from high-power water cooled lasers, to laser diodes in various wavelength regimes. The energy radiated by fluorescent emission and scattering may be processed by photo-detectors, or spectrum analyzers, be analog-to-digital converted (ADC) for processing, and be stored and analyzed by a computer system.

Fluorescently-tagged antibodies may be used to determine cell phenotypes based on surface markers to which the tagged antibodies become attached. The fluorescent materials tend to have a rather broad emission wavelength distribution so that emissions associated with one antibody are detected in a wavelength channel associated with another antibody, particularly if the signal of the interfering antibody is strong with respect to another antibody. These effects need to be corrected by calibration measurements using each of the antibodies, and may limit the number of antibodies which may be tested for in each experiment.

The optical properties of gold and silver nanoparticles depend on both the particle size and shape and are related to the interaction between the metal conduction electrons and the electric field component of the incident electromagnetic radiation, which leads to strong, characteristic absorption in the visible to infrared region of the spectrum. In aqueous solutions, gold nano-structures exhibit strong plasmon bands depending on their geometric shape and size. For spherical particles, for example, a strong absorption band around 520 nm due to the excitation of plasmons by incident light can be readily observed. For nanorods, two distinct plasmon bands, one associated with the transverse (˜520 nm) mode and the other with the longitudinal mode (usually >600 nm) may be observed. Plasmon modes have also been reported for more complex structures such as prisms and quadrupoles.

### SUMMARY

An apparatus for measuring the relative expression of cell surface markers is disclosed, including an optical system adapted for imaging a cell having a functionalized probe attached onto an optical spectrum analyzer. A darkfield illumination device may be configured to illuminate the cell, and the bandwidth of an optical illumination source is greater than an absorption spectrum bandwidth of the probe. An optical radiation spectrum of the functionalized probe is obtained.

A method of measuring cell characteristics includes the steps of providing a first and a second type of functionalized probe; incubating the functionalized probes with a cell sample having a plurality of cells; and measuring a spectrum of optical energy re-radiated from a cell having an attached functionalized probe of at least one of the first or the second type of functionalized probe. A first probe type is functionalized so as to, for example, attach a first cell marker and a second probe type is functionalized to attach to a second cell marker.

In another aspect, a contrast agent for imaging studies is disclosed, including, a gold nanoparticle; a magnetic nanoparticle bound to the gold nanoparticle; and an antigen bound to the gold nanoparticle.

In yet another aspect, a drug delivery compound is disclosed, the compound including a gold nanoparticle, having a biomarker bound thereto; a magnetic particle bound to the gold nanoparticle; and a therapeutic drug bound to the magnetic particle.

A computer program product, stored on a computer readable media, includes instructions for configuring a computer to accept data from a spectral imaging device; determine a number of cells of a population of cells having one or more functionalized probes types attached thererto; and, using one of the functionalized probe types as a reference, computing the relative abundance of at least one immunophenotype with respect to the abundance of a reference immunophenotype.

In another aspect, a method of providing a targeted contrast agent for imaging studies of a patient is described, the method including providing a functionalized probe having an antigen for a specific surface marker, and a magnetic particle; and, administering the functionalized probe to the patient.

In another aspect, a method of providing targeted drug delivery to a patient is described, the method comprising, providing a functionalized probe having an antigen for a specific cell surface marker, and a particle having a therapeutic drug bound thereto; and, administering the functionalized probe to a patient having cells characterized by a surface protein bindable to the functionalized probe.

### BRIEF DESCRIPTION OF THE DRAWINGS

FIG. 1 is a plot of the measured extinction of light scattered from gold nanorods as a function of wavelength, for gold nanorods of aspect ratio 2.8, 3, 4.5, 5.5, and 7;

FIG. 2 shows a representation of a method for functionalizing nanorod probes;

FIG. 3 shows an experimental apparatus for measuring the characteristics of functionalized probes attached to cells of a cell sample;

FIG. 4 shows dark field images of three HBEC lines: a. MCF10A; b. MDA-MB-436; c. MDA-MB-231; and, d. mean measured spectrum;

FIG. 5 shows darkfield images and plasmon spectra of cells of different immunophenotypes obtained using GNrMPs having three different aspect ratios (1.5, 2.8 and 4.5): a. CD24−/CD44− (GNrMP 598:CXCR4; GNrMP690:CD24; GNrMP829:CD44); b. CD24+/CD44+; c. CD24+/CD44−; and, d. CD24−/CD44+;

FIG. 6 shows GNrMP plasmon spectra (aspect ratios 1.5, 2.9, 4.5) of MBA MD231 cells with immunophenotypes of: a. CD49f/CD44+/CD24−; b. CD49f/CD44+/CD24+; and, c. CD49f/CD44−/CD24−; and

FIG. 7 is a flow chart of a method of using functionalized probes to determine the relative abundance of immunophenotypes in a cell population.

### DETAILED DESCRIPTION

Reference will now be made in detail to examples. While the invention will be described in conjunction with these examples, it will be understood that it is not intended to limit the invention to such examples. In the following description, numerous specific details are set forth in order to provide a thorough understanding of the present invention which, however, may be practiced without some or all of these specific details. In other instances, well known process operations have not been described in detail in order not to unnecessarily obscure the description.

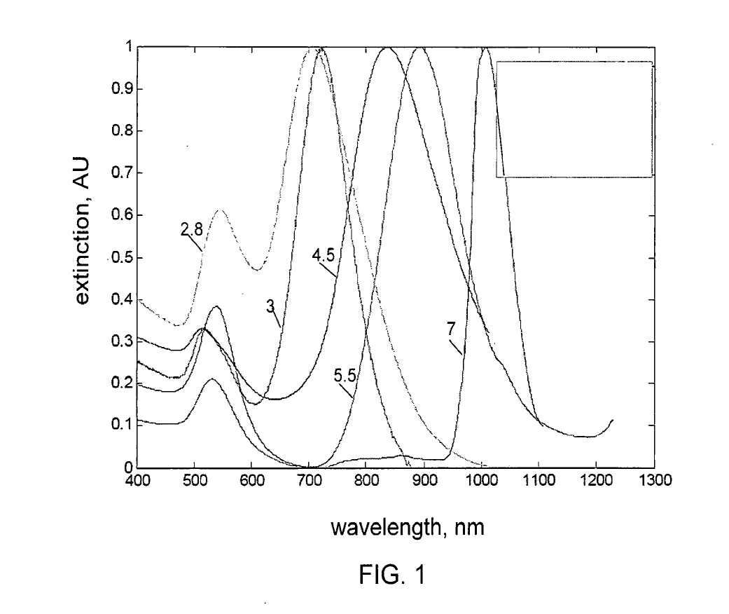

FIG. 1 shows the absorption spectra of gold nanorods (GNR) with aspect ratios of 2.8, 3, 4.5, 5.5, and 7, respectively. Small changes in aspect ratio introduce a significant red-shift in the longitudinal plasmon (LP) band of the GNR colloids. Within the above range of aspect rations, a substantially linear relationship between the aspect ratio of gold nanoparticles of rod-like form and the absorbance wavelength of the longitudinal plasmon bands may be observed; hence, the aspect ratio of gold nanorods may be deduced from their plasmon spectra. As such, a nanorod having a first biosensor molecule functionalized thereto may be distinguished from a nanorod having a second biosensor molecule attached thereto by measuring the plasmon spectrum thereof. Other materials exhibiting similar optical properties, such as silver, may also be used. In addition, multifunctional nanomaterials, such as magnetic particles attached to gold nanorods may also be used.

Probe particles may be functionalized, for example, using antibodies for specific surface markers expressed in cells. The functionalized probes may be observed using, for example, spectral imaging, and the quantity, intensity, or types of probes which have attached to the cells may be measured so as to indicate the abundance of one or more specific surface markers.

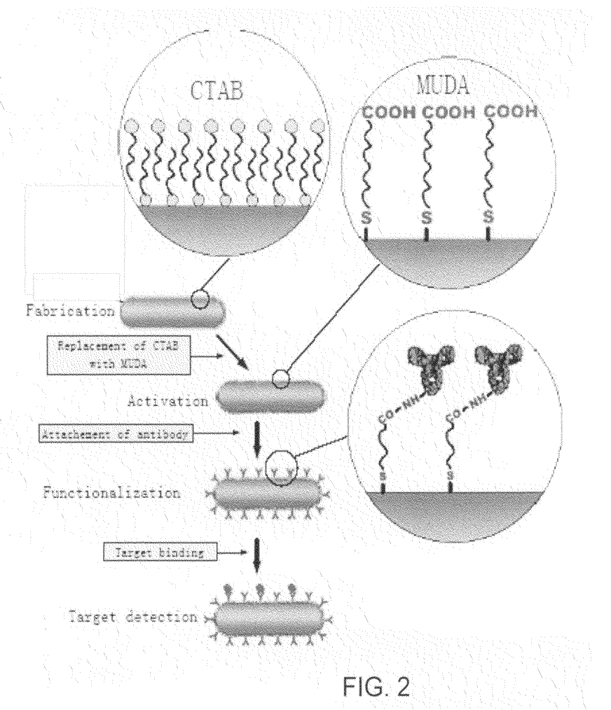

As shown in FIG. 2, biofunctionalization may be a two step process: in step 1, termed as the activation step, a chemical anchor layer may be formed on a nanorod surface to provide active functional groups to which biological molecules (e.g., antibodies) can be attached; and in step 2, the functionalization step, biomolecules may be covalently linked to the anchor layer to produce nanoparticle molecular probes for target specific sensing. The process of biofunctionalization may thus result in a biological molecule being attached to the nanorod or probe. The functionalized probe may be, for example, a gold nanorod functionalized with a biological molecule (GNrMP) and may be used to detect, for example, a marker molecule that is expressed on a cell surface.

GNrMPs are non-bleaching and may be used in a method to rapidly interrogate cells, which may be living cells, for identity-profiling under physiological conditions. As an example, the method was used to profile three human breast epithelial cell lines with different malignancy and metastasis status (MCF10A, MDA-MB-436 and MDA-MB-231); and, the presence of subpopulations of cells with different immunophenotypes and combinations thereof was determined in the cell lines. Differences in the immunophenotypic composition of the cell lines were observed across cell lines and may be correlated with the invasiveness and metastasis potential of the 3 cell lines.

Gold nanorods with several different aspect ratios were prepared by a wet-chemistry, seed-mediated growth method. Three sizes of gold nanorods, having aspect ratios of 1.5, 2.8 and 4.5, were used for the synthesis of GNrMPs. The seed mediated growth procedure produces gold nanorods with a CTAB (Hexadecyltrimethylammoniumbromide (C₁₆TAB, 99%) coating. CTAB is known to be cytotoxic. To functionalize the probes for in vivo use, the CTAB caps were removed by elevating the temperature of the solution having an alkanethiol, while the gold nanorods were kept from aggregation by sonication.

11-mercaptoundecanoic acid (MUDA) was used as an alkanethiol to react with gold nanorods to produce an activated surface for biofunctionalization. The nanorods were suspended in water at 20 nM, to 5 ml of the solution, 1 ml of 20 mM MUDA in ethanol was added and the solution was kept at 60° C. under constant sonication for 30 minutes. Then, the temperature was decreased to 30° C. and the solution was maintained under constant sonication for 3 hours. The solution was then subjected to chloroform extraction for three rounds and the gold nanorods were collected by centrifugation and re-suspended in PBS (Phosphate Buffer Saline) (pH 7.4, Sigma).

After activation, the gold nanorods were functionalized with antibodies against CD24, CD44, CD49f (Pierce Biotechnology, Inc., Rockford, Ill.) and CXCR4 (R&D Systems, Minneapolis, Minn.). 2.5 ml of activated nanorods (20 nM) was treated with a mixture of 1-Ethyl-3-[3-dimethylaminopropyl]carbodiimide hydrochloride (EDC) (0.4 M) and N-hydroxysuccinimide (NHS) (0.1 M), and then incubated with the antibody solution at 0.1 μM at 4° C. under sonication for 30 minutes. The unbound antibodies were removed by centrifugation and the remaining free binding sites on the nanorod surface were blocked by treating the nanorods with 0.1 M ethanolamine solution. The functionalized gold nanorod molecular probes (GNrMPs) were then re-dispersed in PBS buffer at 10 nM and stored at 4° C.

The GNrMPs produced by this method appeared to remain stable for up to about at least 100 days without significant aggregation; the wavelength of the plasmon bands experienced no apparent change within this time period. The intensity of the plasmon bands exhibited a small drop in intensity value after 30 days due to minor aggregation, but the change of plasmon band intensity was less than 5% after even 100 days.

Monodispersed magnetic nanoparticles were modified to bear COOH groups using the following a known procedure: 2 mM of Fe(acac)3 was dissolved in a mixture of 10 mL benzyl ether and 10 mL oleylamine. The solution was dehydrated at 110° C. for 1 h, quickly heated to 300° C., and kept at this temperature for 2 hours. 50 mL of ethanol was added to the solution after the solution was cooled down to room temperature. The precipitate was collected by centrifuging at 8000 rpm and then washed by ethanol 3 times and the product was re-dispersed in hexane. To this, 1.7 mg of dopamine hydrochloride dissolved in a mixture of (trichloromethane) CHCl₃ (2 mL), (N,N-Dimethylformamide) DMF (1 mL), and then Fe₃O₄ nanoparticles (5 mg) were added. The resulting solution was stirred overnight at room temperature under N₂. The amino modified Fe₃O₄ nanoparticles were precipitated by adding hexane, and magnetically separated and dried under N₂. 5 mg of this amino modified Fe₃O₄ nanoparticles were dissolved in 200 mL, 10 mg/mL anionic poly(acrylic acid) (PAA). The reaction time was kept at 2 h. The resulting solution was centrifuged three times to remove excess PAA to obtain COOH modified Fe₃O₄ nanoparticles.

Attachment of COOH modified magnetic particles to amine terminated gold nanorods may be then accomplished through EDC/NHS (1-ethyl-3-[3-dimethylaminopropyl]carbodiimide hychloride/N-hydroxysuccinimide) coupling chemistry.

In an aspect, the fabricated gold nanorods terminated with amine groups may then be converted to gold nanorod molecular probes by attaching cancer cell surface marker antibodies to these nanorods using the procedure previously described.

Magnetic particles attached to antibody-functionalized-gold-nanorods may, for example, be used to capture cells bearing these specific markers directly in blood and be magnetically separated and placed on cover slips and tested using a spectral scanning imager similarly to the GNrMP-based detection. Magnetically functionalized gold nanorod particles may also be used to perform organism profiling. In an aspect, biomarkers relevant to proteins, RNA, and DNA in, for example, formalin fixed paraffin-embedded frozen tissue from patient biopsy may be selected for functionalization of the gold nanorods. Using a cross-platform method, markers for protein, DNA and RNA, for example, may be contemporaneously detected.

Magnetic nanoparticles may be selectively assembled onto the ends and ends and sides of gold nanorods of different aspect ratios (AR) to fabricate multifunctional nanoparticles. The resulting hybrid nanoparticles may be described, for example as Fe₃O₄—Au_{rod}—Fe₃O₄ nanodumbbells (for example, two Fe₃O₄ tips) and Fe₃O₄—Au_{rod} pearl-necklaces (many Fe₃O₄ nanoparticles around a gold nanorod), respectively. Such hybrid nanomaterials having both NIR (near infrared) optical and magnetic properties have been prepared by controlling the reaction conditions. Site-selective assembly of magnetic nanoparticles to gold nanorods may be used to tune the optical and magnetic properties of hybrid nanoparticles. Such particles can be used to separate and detect cancer cells, pathogenic microorganisms, and other hazardous agents.

A nanorod linked to magnetic particles may have the ability to carry two or more different attached biomolecules. An antibody can be attached to the amine terminated nanorods using the glutaraldehyde procedure while a drug (for example. Taxol or Doxorubicin) can be attached to the carboxyl group of the magnetic particles. The compound may be used, for example, to target a tumor site based on the antibody attached to the nanorod part of the nanorod-linked-magnetic-particle of the functionalized probe. Once the probes lodge on to the tumor site, the attached drug is deliverable to the tumor site. The magnetic particles assembled to nanorods attached to antibodies can be used target a cell based on the cell surface marker specificity and delivery a drug.

In yet another aspect, the magnetic particles attached to the functionalized gold nanorods may be employed as contrast agents in medical imaging studies using, for example, magnetic resonance imaging (MM), where the biomarkers bond to sites on the cell surface, and the magnetic particles act as contrast agents specific to the cell immunotype. Similarly, the magnetic particles may be used to deliver drugs to a specific cell type as defined by the biomarker, and as a plurality of magnetic particles may be attached to a gold nanorod, the amount of drug or contrast agent delivered is increased. The particles may also be used for optical detection using Raman microscopy. The functionalized probes may be administered to patients in the same manner as is known for the delivery of contrast agents for imaging or drugs for treatment of disease.

Gold nanorods with different ARs were prepared. Preferential binding of cetyltrimethyl ammonium bromide (CTAB) bilayers along the {100} facet of the longitudinal side of the gold nanorods left their ends (the {111} faces) deprived of CTAB and allow for the selective binding of cystamine dihydrochloride (abbreviated in cystamine), water soluble with a disulfide and two amino groups, to the ends of gold nanorods, resulting in partially activated gold nanorods with amine groups at room temperature. In addition, to obtain completely activated gold nanorods with amine groups, that is, NH₂ groups provided by cystamine self-assembled onto the ends (the {111}faces) and sides (the {100} faces) of gold nanorods, and elevating the solution reaction temperature, may cause CTAB to disassociate form the {100} faces of gold nanorods, leading to amino modified gold nanorods.

Monodispersed Fe₃O₄ nanoparticles capped with carboxyl groups were synthesized using the procedure described previously. The carboxyl-terminated magnetic nanoparticles were selectively assembled onto the ends and sides of partially and completely amine modified gold nanorods by 1-ethyl-3-[3-dimethylaminopropyl]carbodiimide hychloride/N-hydroxysuccinimide (EDC/NHS) chemistry. The resulting hybrid nanoparticles are the Fe₃O₄—Au_{rod}—Fe₃O₄ nanodumbbells and Fe₃O₄—Au_{rod} pearl-necklaces.

Since a plurality of magnetic particles may be attached to a hybrid nanoparticles or probes the nanodumbbell, the nanopearl necklace, or the like, is a way of concentrating magnetic particles so that the particles can serve as more efficient Magnetic Resonance Imaging (MRI) contrast agents, or drug delivery agents, than unlinked magnetic particles.

The details of the procedure may include the steps of (a) synthesis of COOH functionalized magnetic nanoparticles; (b) synthesis of gold nanorods with different aspect ratios; (c) selective modification of gold nanorods with NH₂ groups; (d) selective assembly of magnetic nanoparticles to NH₂ modified gold nanorods; and, (e) biofunctionalization of Fe₃O₄—Au_{rod} pearl-necklaces with antibodies. In more detail, the steps may include:

(a) Synthesis of COOH functionalized magnetic nanoparticles: Uniform 15 nm magnetic nanoparticles capped with oleic acid were synthesized by a known procedure. The surfaces of magnetic nanoparticles were functionalized with COOH groups by coating amphiphilic polymers; (b) Synthesis of gold nanorods with different aspect ratios: CTAB-stabilized gold nanorods were synthesized using the seed-mediated growth method. The seed solution was prepared by mixing of CTAB (0.2 M, 5 mL) and HAuCl₄ (0.5 mM, 5 mL) with freshly prepared ice-cold NaBH₄ (10 mM, 0.6 mL). After 5 hours, this seed solution was used for the synthesis of gold nanorods. In two flasks, 50.0 mL of 0.2 M CTAB was mixed with different amounts of 10 mM silver nitrate (30 μL and 120 μL, respectively) and 50.0 mL of 1 mM HAuCl₄. After gently mixing the solution, 600 μL of 0.10 M ascorbic acid was added. Then, 120 μL of the seed solution was added into the mixture to initiate the growth, and yielded gold nanorods of aspect ratios 2.0 and 3.3. After preparation, the excess CTAB was removed by centrifuging twice at 8000 rpm, and discarding the supernatant and re-dispersing the particles in pure water; (c) Selective modification of gold nanorods with NH₂ groups: NH₂ group modification of the ends of gold nanorods was carried out by adding a calculated volume of cystamine (0.01 M) into the as-prepared original gold nanorod solution leading to a cystamine concentration of about 100 μM. The resulting solution mixture was kept at room temperature for 3 h. NH₂ group modification of the ends and sides of gold nanorods was achieved as follows: 0.5 mL of 20 mM aqueous solution of cystamine was added into 5 mL of gold nanorod solution and centrifuged once and sonicated for 3 h at 50° C. The resulting gold nanorods were then collected by centrifugation twice at 7000 rpm for 15 min in order to remove excess cystamine and CTAB and re-suspended in a 0.005 M CTAB solution to yield a final concentration of 100 nM; (d) Selective assembly of magnetic nanoparticles to NH₂ modified gold nanorods: 0.1 ml of 5 mg/mL (Fe)COOH modified Fe₃O₄ nanoparticles was mixed with 1 mL pH 5.5 10 mM borate buffer, then 100 uL of 1 mg/mLEDC and 50 ul of 1 mg/mL NHS were added to Fe₃O₄ with sonication at 4° C. for 15 min. NH₂ modified the ends of gold nanorods and ends and sides of gold nanorod solution were added to the activated Fe₃O₄ nanoparticle solution by EDC and NHS, respectively, and sonicated for 30 min and the resulting solution were centrifuged at 4000 rpm to remove unbound magnetic nanoparticles. Subsequently, the free gold nanorods unattached to magnetic nanoparticles were separated in the presence of magnet outside the container; (e) Biofunctionalization of Fe₃O₄—Au_{rod} pearl-necklaces with antibodies: Fe₃O₄—Au_{rod} pearl-necklaces with some remaining NH₂ groups were dispersed into phosphate-buffered saline (PBS, Na₂HPO₄ 8.1 mmol/L, NaH₂PO₄ 1.9 mmol/L, NaCl 1.4 mol/L, Tween-20 0.05%, pH 7.4) containing 5% glutaraldehyde for about 1 hour at room temperature. The hybrid nano particles were collected by centrifugation and re-dispersed in PBS, and then incubated with antibodies for about 3 hours at 37° C. The antibody-modified Fe₃O₄—Au_{rod} hybrid nanoparticles were then washed with PBS to remove the excess antibody and kept at 4° C. in PBS.

Chemicals were obtained from Sigma Aldrich, St. Louis, Mo.

In an experimental example of the use of the particles functionalized with antibodies, nonmalignant human breast epithelial cell (HBEC) line MCF10A and two malignant HBEC cell lines (MDA-MB-436 and MDA-MB-231) were cultured on 18 mm diameter glass cover slips in a 6-well tissue culture plate. MCF-10A cells were grown in DMEM/F12 media containing 5% horse serum and the following supplements: 10 μg/ml insulin, 20 ng/ml epidermal growth factor, 100 ng/ml cholera euterotoxin, 0.5 μg/ml hydrocortisone and 2 Molar (m)/1 L-glutamine. MDA-MB-436 and MDA-MB-231 cells were cultured in DMEM with 10% FCS. The cell cultures were incubated at 37° C. under 5% CO₂. Once the cells reached confluence (48˜72 hours), 0.5 ml of 10 nM gold nanorod molecular probes (a mixture of all three types) was added to the media and incubated for 30 minutes at 4° C. to allow binding to the respective cell surface markers. The cells on the cover slips were then rinsed with PBS buffer and sealed with a microscope glass slide with pre-etched chambers containing 100 μl of fresh medium to keep the cells moisturized and in their physiological state. Cells may live for up to at least about 1 hour under these conditions.

The attachment of GNrMPs to cell surface markers was confirmed through back-scattering field emission scanning electron microscopy (FEI NOVA nanoSEM field emission SEM, FEI Co., Hillsboro, Oreg.), where CD44/CD49f GNrMPs binding to MDA-MB-231 cells were visualized (FIG. 2). Cells were reacted with GNrMPs for 30 minutes and then washed vigorously using 0.1 M K—Na₂-Phosphate buffer (pH 7.4) to remove unbound GNrMPs. The remaining GNrMPs appeared as individual probes binding to cell surface markers. Some of the GNrMPs appeared as larger clusters on the cell surfaces. The direct observation of GNrMPs bound to the cell surface demonstrates the efficacy of the GNrMP process.

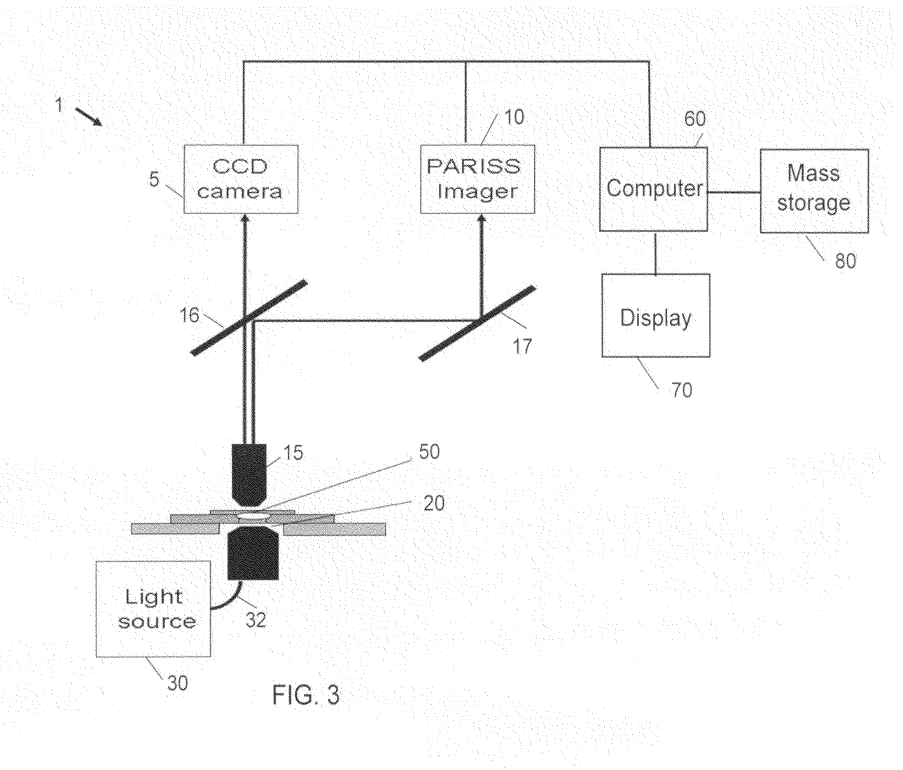

The reacted cells were visually observed using a darkfield microscope, and the effect of the plasmon resonance was observed using a prism and reflector imaging spectroscopy system spectroimager (PARISS) by hyperspectral imaging. In the experiment, the two sensors were combined so as to use a single microscope, and a beam splitter, as shown in FIG. 3.

Darkfield imaging of a sample is performed by illuminating the sample in a manner such that the direct illumination of the detector or imaging device, by the illuminating source is avoided. This is often done by occluding a portion of the light beam in the field of view of the lens, and focusing the remaining light to pass through the sample such that only the scattered light from the sample is imaged onto the detector. The bandwidth and central wavelength of the light source should be compatible with the wavelength range of the GNrMPs to be measured.

The spectral characteristics of the scattered light may be observed by any of a variety of known types of optical spectrum analyzers. The spectral data may be obtained using multiple bandpass filters, sequential spectrum analysis using, for example, diffraction gratings in conjunction with a photodetector, multispectral analysis using a diffraction grating in conjunction with a spatially dispersed photodetector detector array such as a charge coupled device (CCD), or wavelength dispersion through a prism. The spectral analysis may also be performed in such a manner that the individual cells passing by a slit may be spatially resolved along the slit and the spectrum for each cell determined individually, for multiple individual cells simultaneously. An example of this type of hyperspectral imaging spectrometer is the PARISS available from LightForm, Inc., Hillsborough N.J. A slit or a flow cell may be used to present the cells for measurement such that the characteristics of the cells may be individually determined.

The reacted cells were visually observed using a darkfield microscope, and the effect of the plasmon resonance was observed by hyperspectral imaging using a prism and reflector imaging spectroimager. The experimental apparatus 1 includes a darkfield illuminating stage 50, having an aperture through which light is directed by a condenser lens 20 so that light scattered by a cell sample may be intercepted by an objective lens 15. The light source, which may be a mercury lamp or other suitable source 30, may be directed to the condenser 20 by a light pipe 32, or other suitable optical arrangement of lenses, reflectors, or the like. The light emerging from the objective lens 15 is divided by a beamsplitter 16 so that a portion of the scattered light is incident on a digital camera 5, which may have a charge-coupled device (CCD) or other photosensitive detector, which may be an array of photodetectors. Another portion of the light may be reflected by the beamsplitter 16, and by a reflector 17 or other optical devices, so as to be incident on an optical spectrum analyzer, which may be a PARISS imager 10, or other type. The apparatus may be arranged so that the spectrum of the incident light scattered by one or more cells may be obtained, and the data converted to a form suitable for input to a computer 60. The computer may have mass storage capability 80 incorporated therein, or as an external capability, and the data may be displayed by a computer display 70. The raw data, the images, and the analyzed data may be displayed, and an operator may control the operation of the apparatus using a keyboard, mouse or other input device (not shown). The computer may have an interface to a local area network so that the data may be transferred to another computer, a data center, or remotely over a wide area network such as the Internet. Although the connections between the electronic devices are shown as cables, connection of the various devices may be by wireless interfaces, as are known or may be developed.)

The combination of hardware and software to accomplish the measurements and analysis operations described herein may be termed a system. The instructions for implementing processes of the system may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

In an embodiment, the instructions may be stored in a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet”, or the like, is used, the intent is to describe an internetworking environment, including both local and wide area telecommunications networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths. Often, the internetworking environment is provided, in whole or in part, as an attribute of the facility in which the platform is located.

Communications between the devices, systems and applications may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions. Such wireless communication may be performed by electronic devices capable of modulating data as signals on carrier waves for transmission, and receiving and demodulating such signals to recover the data. The devices may be compatible with an industry standard protocol such as IEEE 802.11b/g, or other protocols that exist, or may be developed.

Darkfield images of the cells may be obtained using an Olympus BX40 microscope as an optical sub-system, equipped with a CytoViva darkfield module (Aetos Technologies, Inc., Auburn, Ala.) for darkfield imaging, where scattering from GNrMPs appears as bright particles against a dark background. Imaging was accomplished through the collection of the scattered light using a 40× microscope objective. The optical sub-system output was also spectrally resolved in a PARISS spectroimager at a spectral resolution ˜2 nm, and detected with a CCD camera to obtain absorption/scattering plasmon spectra of gold nanorod molecular probes attached to the cell surfaces. This may allow the prepared samples to be scanned at a speed of about 60 mm/s, which may correspond to about 0.5 ms/cell. Regardless of the number of differing aspect ratio GNrMPs used, the data may obtained for all of the functionalized GNrPM types at the same time.

GNrMPs designed for different cell surface markers bind to their targets on the cell surface to absorb optical signals which may be detected using the PARISS imager. The ratios of extinction values for two or more GNrMPs, each having an aspect ratio associated with a cell surface marker, may be used to determine the relative abundance of the surface markers in a population of cells.

In an aspect, a surface marker that is ubiquitously expressed across all the cell types at RNA level may be used as an reference value so that the data may be quantitatively compared. In previous studies of thirteen breast epithelial cell lines, the expression of CXCR4 was found to be the highest at the RNA level as measured by Northern blotting in TMD 231 cells. The cell lines investigated in the examples described show similar expression level. Hence, measured CXCR4 abundance may be used as self-reference to evaluate the expression levels of other markers (such as, CD24, CD44, and CD49f).

At present, however, the sensitivity of flow cytometry techniques, may only allow the detection of the cell surface expression of CXCR4 protein in the most highly expressed cells (TMD-231 cells), whereas the GNrMP assay procedure described herein yields detectable CXCR4 signals in cell lines that may not be possible otherwise. As a consequence of CXCR4 in the cell types used in this experiment, signals from other surface markers (CD24, CD44, CD49f) can be semi-quantitatively evaluated based on their relative intensity to the reference; therefore, the expression levels of these markers, which are proportional to the signal intensity of their respective GNrMPs, can be estimated.

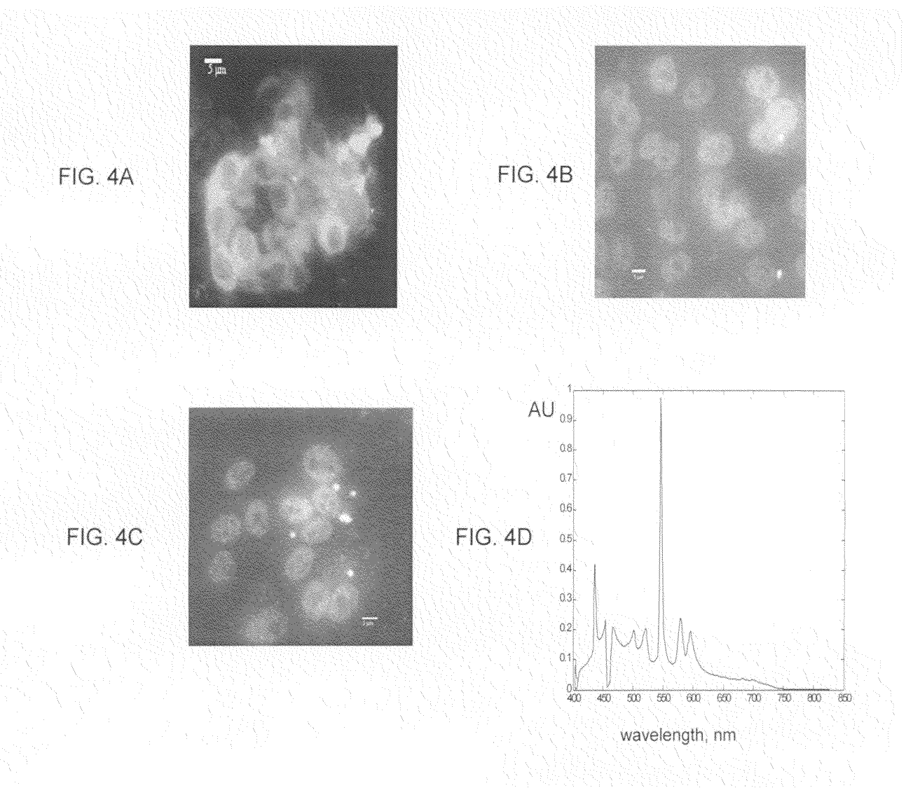

FIGS. 4a-c show dark field images of MCF10A, MDA-MB-436 and MDA-MB-231 cells, respectively, with no GNrMPs attachment. Since light is scattered differently by the nucleus and cytoplasm of the cell, a good contrast ratio was observed. The morphological characteristics of the three cell line types appear quite similar and do not appear to be usable as a criterion for differentiation among the cell lines, especially for the two malignant cell lines (MDA-MB-436 and MDA-MB-231). When the optical signals were analyzed by the PARISS imager, the characteristic white-light spectrum of the mercury lamp is obtained, as shown in FIG. 4d. The scattering and absorption of cells did not alter the spectral characteristics of the transmitted light significantly.

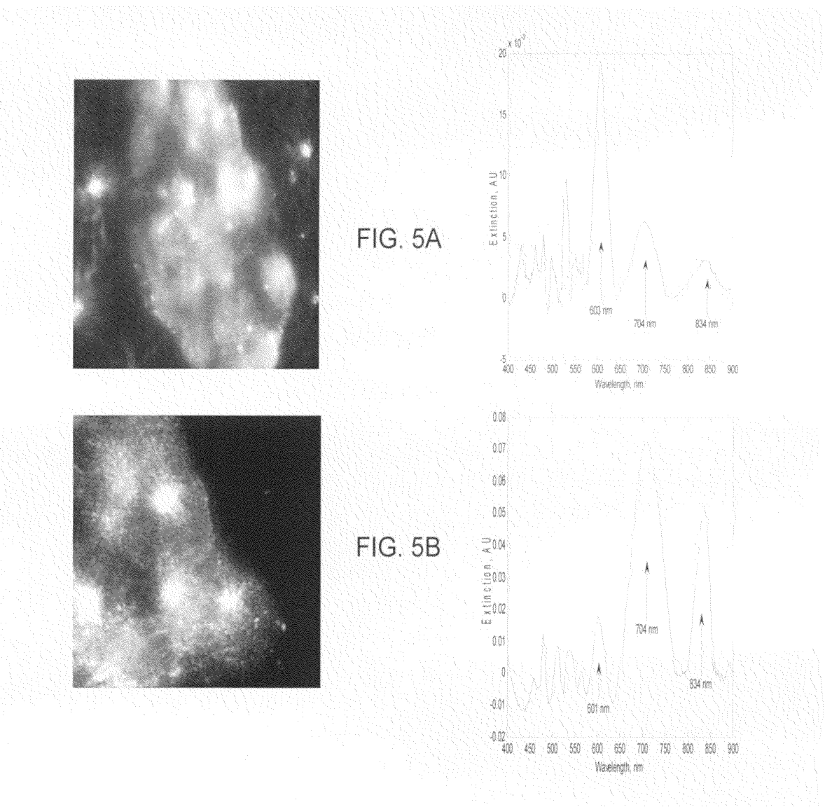

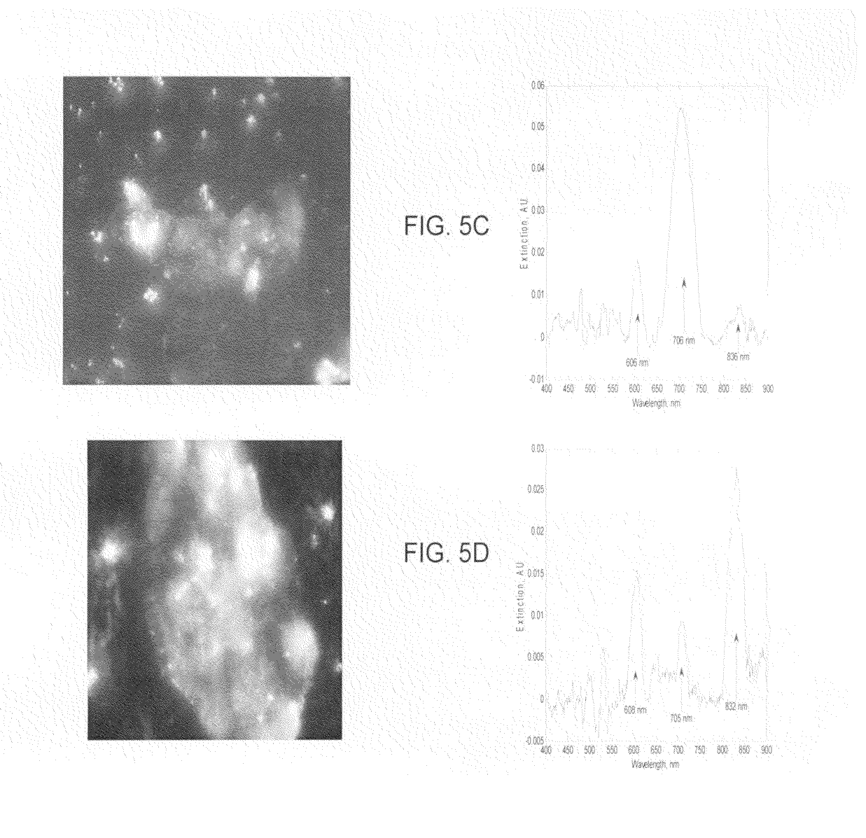

When GNrMPs were incubated with cell samples having known biomarker targets, substantially different darkfield and spectral characteristics were observed, which may arise from the binding of GNrMPs to the cell surface marker targets. FIGS. 5a-d shows the darkfield images of MCF10A cells of different immunophenotypes with the attached GNrMPs and their respective spectra measured by the PARISS spectral imager. In FIG. 5a, the contrast between cell nucleus and cytoplasm can be observed by visual inspection, while stronger scattering of light from some GNrMPs is also clearly seen. The observed spectrum exhibits significant changes; three spectral bands corresponding to the three GNrMPs indicative of the markers may be identified within the spectrum. Thus, a plurality of cell biomarkers may be simultaneously observed and measured so as to characterize the cell.

The CXCR4 band is the most intense; the CD24 and CD44 bands are both weaker, suggesting a lower expression level for these two markers as compared to CXCR4. As CXCR4 itself is expressed at a low to moderate level in HBEC cells, this observation indicates that cells shown in FIG. 5a displayed the immunophenotype, CD44−/CD24−.

Visual inspection of FIG. 5b does not appear to reveal any detailed sub-cellular structures; the image appears to be dominated by strong scattering light from GNrMPs, suggesting the presence of relatively large numbers of GNrMPs on the cell surfaces. The spectral analysis is consistent with strong signals originating from CD44 and CD24 tethered GNrMPs, compared to the moderately expressed reference of CXCR4, suggesting an immunophenotype of CD44+/CD24+. When compared to the results of FIG. 5a, the binding of GNrMPs to the cell surface does not appear to be explained by non-specific interaction between the GNrMPs and the cells, which further suggests the immunophenotype as CD44+/CD44+ for these cells. In FIGS. 5c and 5d, immunophenotypes of CD44−/CD24+ and CD44+/CD24− were observed, indicative of the higher expression levels of CD24 and CD44 cell surface markers, respectively.

Another experimental observation was that the GNrMP signals showed a spectral red-shift in the longitudinal plasmon bands upon binding to cell surfaces. The red shift observed varied from 3˜16 nm for different GNrMPs and different cell immunophenotypes. These shifts may be caused by changes in the dielectric environments of the GNrMPs upon binding to cell surface markers; the scale of the shift may be useable to quantitatively evaluate the binding affinity of GNrMPs to their targets in a multiplex format. To the extent that the binding affinity of a GNrMP to a cell surface marker may be dependent on the aspect ratio of the GNrMP, the measurements may be repeated where the association of the cell marker with an aspect ratio is changed.

The experimental results show that, in MCF10A cells, four immunophenotypes of CD44+/CD24+, CD44−/CD24−, CD44−/CD24+ and CD44+/CD24− are present. By counting the numbers of each immunophenotype in a cell population, the immunophenotype composition of MCF10A cells listed in Table 1 was determined. In MCF10A cells, the most dominant immunophenotype CD44−/CD24− constitutes 62.7% of the cell population; the highly invasive immunophenotype CD44+/CD24− constitutes about 14.3% of the cell population, suggesting that MCF10A cell line may not be a highly invasive cell line. When MDA-MB-436 and MDA-MB-231 cells were investigated, a different pattern was observed. As listed in Table 1, in these two cell lines, CD44+/CD24− cells are the most dominant constituting 84.4% and 72.1% of the cell population, respectively while the CD44−/CD24+ does not appear to be observed.

The immunophenotype composition of the cell population acquired by the GNrMP assay was validated by flow cytometry analysis and also presented in Table 1. The GNrMP results are in good agreement with flow cytometry results, suggesting that the GNrMP assay is a suitable method for cell identity-profiling.

TABLE 1
Immunophenotype composition of cell population of three HBE cell lines

| | CD24+/CD44+ | | CD24−/CD44− | | CD24−/CD44+ | | CD24+/CD44− | |
|---|---|---|---|---|---|---|---|---|
| | Scanner | Cytometry | Scanner | Cytometry | Scanner | Cytometry | Scanner | Cytometry |
| MCF10A | 6.6% | 5% | 62.7% | 58% | 14.3% | 17% | 16.97% | 20% |
| MDA MB 436 | 19.8% | 22% | 8.1% | 7% | 72.1% | 71% | 0% | 0% |
| MDA MB 231 | 3.5% | 2% | 12.1% | 13% | 84.4% | 85% | 0% | 0% |

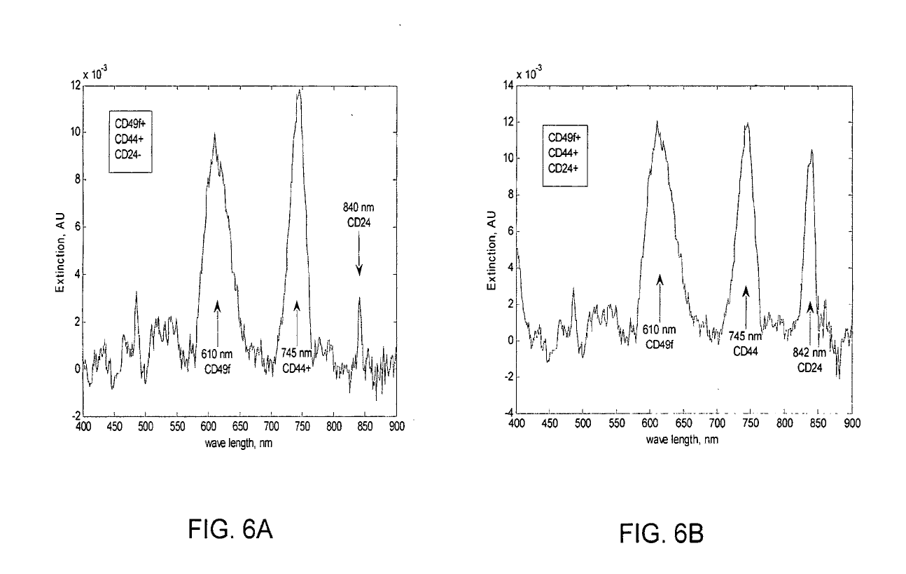

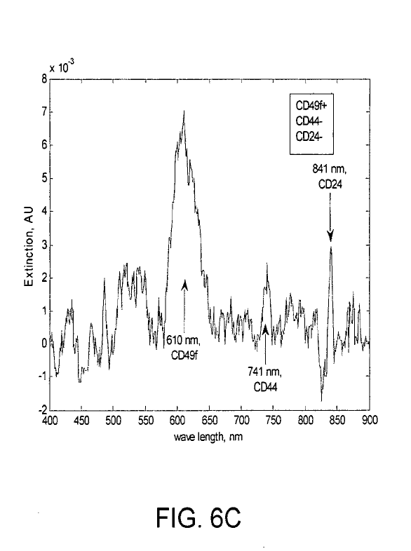

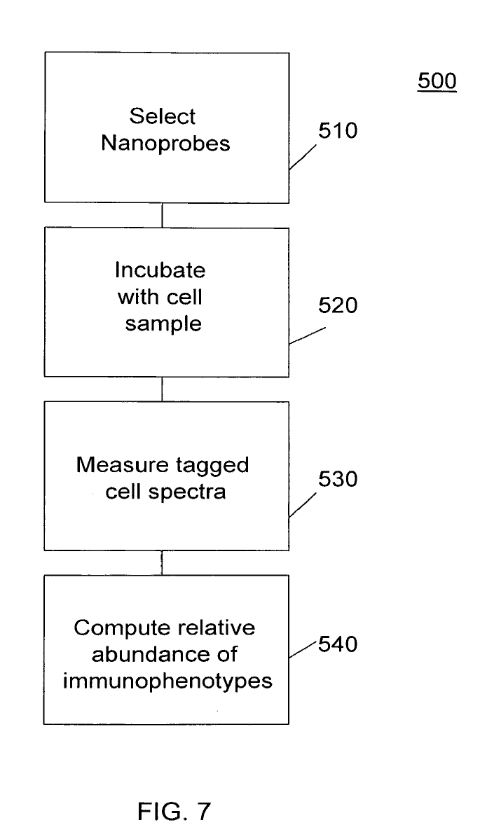

In addition to CD24 and CD44, CD49f is another cell surface marker that has been found to be associated with the sternness of breast epithelial cells GNrMPs with anti-CD49f markers were used to investigate the expression of CD49f in CD44+ and CD44− cells. In MDA-MB-231 cells. CD44+ immunophenotypes constitute 88% of the cell population (3.5% CD24+; 84.4% CD24−). In the CD44+ immunophenotype, CD49f was observed in both CD24+ and CD24− cells, at a relatively high expression level compared to CD44, as shown in FIGS. 6a and b; while in CD44− immunophenotype, the expression level of CD49f seems to be lower than in CD44+ cells (FIG. 6c). These observations suggest that there may be a correlation between high expression levels of CD49f and CD44.

The experimental examples of use of the method indicate that the relative abundance of cell surface markers can be measured by nanorod-based bioprobes. In addition, different cell surface markers can be simultaneously measured by nanorods of different aspect ratios because of the unique spectral peak corresponding to the aspect-ratio-dependent nanorods.

The examples described used specific antigens as biomolecules that were believed to be appropriate for the experiment performed, as well as for a surface protein expressed substantially uniformly in the population of cells having the differing surface protein markers which were being evaluated. Other proteins having substantially uniform expression in a cell type being investigated may be used as a reference, and other antigens appropriate to the phenotypes being investigated may be used, as appropriate, using the same or similar methods of preparation, use, measurement, or analysis.

The number of individual biomarkers that may be used simultaneously is greater than the number used in the experiments described. The upper limit on the number of antigens that may be used depends on factors such as the number of GNR aspect ratios that can be produced, the resolution of the hyper spectroimager, and the relative abundances of the specific antigens being investigated. In addition to simple rod-like shapes, more complex shaped GMRs may be used.

As gold may nanoparticles exhibit resonant scattering by surface plasmon excitation by an optical signal in the range of about 600 to 2000 nm, perhaps 30 distinct probes may be simultaneously accommodated in this spectral region, with a 50 nm spacing. A greater number may be possible.

In another aspect, Fe₃O₄—Au_{rod} pearl-necklace based on gold nanorods with different ARs may be useful for pathogen or tissue type detection applications. As an example an E. coli and S. typhimurium antibody conjugated Fe₃O₄—Au_{rod} pearl-necklace based on gold nanorods of ARs of about 2.0 and about 3.4 was developed to demonstrate two Fe₃O₄—Au_{rod} pearl-necklace bioprobes for detecting multiple bacteria targets. These two antibody-labeled Fe₃O₄—Au_{rod} pearl-necklaces and two species of bacteria were mixed together in the PBS buffer and incubated for 30 min.

The UV-visible absorbance spectra obtained from samples that contained both E. coli and S. typhimurium at different concentrations in the range from 1-10 to 10⁵ cfu/mL (Colony Forming Units/mL) was measured. The LP (longitudinal plasmon) band intensity of the two Fe₃O₄—Au_{rod} pearl-necklace reduced and remained less than that for the LP bands with the addition of E. coli and S. typhimurium. The results indicate that each Fe₃O₄—Au_{rod} pearl-necklace bioprobe based on two ARs could bind to their own target bacteria in a mixture of the two species to result in and intensity reduction of the LP bands of Fe₃O₄—Au_{rod} pearl-necklace bioprobes. This may be because the E. coli and S. typhimurium are much larger in size (˜1-3 μm) than the Fe₃O₄—Au_{rod} pearl-necklace (˜80 nm) modified by anti-E. coli and S. typhimurium antibodies so that two Fe₃O₄—Au_{rod} pearl-necklace bioprobes can be attached to the E. coli and S. typhimurium surfaces. The LP band decreased in intensity after the recognition event between anti-E. coli and S. typhimurium antibodies and E. coli and S. typhimurium. The decrease in intensity reduction at different concentrations from 1-10 to 10⁵ cfu/mL may be due to the interaction of Fe₃O₄—Au_{rod} pearl-necklace bioprobes attached to E. coli and S. typhimurium surfaces. The results indicate that E. coli and S. typhimurium at very low concentrations, for example, less than 10² cfu/mL, can be detected by reduction in LP intensity in less than 30 min. The detection method demonstrated is rapid, since the LP-band reduction of Fe₃O₄—Au_{rod} pearl-necklace bioprobes changes can be observed by using a simple optical spectrometer (for example, a uv-V is NIR spectrometer such as a Lambda XLS from Perkin Elmer, Waltham, Mass.).

In yet another aspect, two Fe₃O₄—Au_{rod} pearl-necklace bioprobes were used as photokilling agents for bacteria. Fe₃O₄—Au_{rod} pearl-necklace bioprobes were allowed to interact with the target bacteria (E. coli and S. typhimurium) for 30 min, followed by magnetic separation. Fe₃O₄—Au_{rod} pearl-necklace bioprobes-bacteria conjugates were then re-suspended in PBS solution. The solution was irradiated with NIR (near infra-red) light (738 nm) at 50 mW for 15 min. The conjugates were then separated and diluted, followed by culturing on a Luria-Bertani (LB) plate for 17 h at 37° C. In the presence of two antibody modified Fe₃O₄—Au_{rod} pearl-necklaces, almost no microorganisms could be observed after NIR irradiation in the plate, while for the two unmodified Fe₃O₄—Au_{rod} pearl-necklaces with antibodies, a number of bacterial cells were observed after NIR irradiation. The Fe₃O₄—Au_{rod} pearl-necklace based on gold nanorods with different aspect ratios absorbed NIR light and converted the light energy into thermal energy to kill the bacteria.

In a further aspect, a method of determining the relative abundance of immunophenotypes in a sample cell population may include; providing a plurality of functionalized probe types, the probes having differing optical properties and each distinct optical property being associated with a immunophenotype. The plurality of functionalized probe types is mixed, or incubated with the cell population. The cells having functionalized probes attached thereto are evaluated using a spectrum analysis technique so as to ascertain the type or types of probes attached thereto, where the probes are identified with immunophenotypes. When one of the functionalized probes is configured to bind to a surface marker that is expressed in all of the cells of the cell population having the other immunotypes, the total abundance of cells may be determined so that relative abundance of cells having various immunophenotypes may be calculated.

In an aspect, the method 500 includes selecting the functionalized nanoprobes (step 510) so that the immunophenotypes of the cell population may be measured; incubating the combination of the cell sample and the nanoprobes (step 520) and so that the nannoprobes may bind to the surface markers of the cells. The incubated sample may be processed so as to remove the unbound nanoprobes. The number of cells having each type of probe attached thereto may be measured by a spectrum analysis technique (step 530). One or more probe types may be bound to each cell, and one of the probe types may have been chosen so that it binds to substantially all of the cells being analyzed. The relative abundance of the immunophenotypes, and combinations thereof may be computed (step 540).

The various probe types may be configured so as to bind to other biological material and be used to detect or identify virus, bacteria, or tissues which may be characterized as phenotypes using the capabilities of the biomolecules of the probes to attach thereto, and a characteristic of the probe such as magnetic material or other contrast agent, plasmon resonance, or the like, so as to identify the presence of one or more phenotypes. For example, multiple pathogentic agents such as E. coli, Salmonella, Listeria, and the like, in food matrices may be identified, quantified, separated, or destroyed.

The examples of diseases, syndromes, conditions, and the like, and the types of examination described herein are by way of example, and are not meant to suggest that the method and apparatus is limited to those described. As the medical arts are continually advancing, the use of the methods and apparatus described herein and adaptations thereof may be expected to encompass a broader scope in the diagnosis and treatment of patients and in research.

Moreover, the apparatus and method may be used to identify other biological material where surface distinguishing features may be present, and for which a probe may be functionalized so as to bind or attach to the surface distinguishing feature or marker.

Moreover, the apparatus and method may be used to identify other biological material where surface distinguishing features may be present, and for which a probe may be functionalized so as to bind or attach to the surface distinguishing feature or marker. In particular, bacteria or tissues may be identified in this manner.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

1. An apparatus for measuring a surface marker; comprising:
an optical system adapted for imaging a cell having a functionalized probe attached thereto onto an optical spectrum analyzer; and
wherein an optical radiation spectrum of the functionalized probe is obtained.

2. The apparatus of claim 1, further comprising:
a darkfield illumination device configured to illuminate the cell; and
an optical illumination source having a spectral bandwidth greater than an absorption spectrum bandwidth of the probe.

3. The apparatus of claim 2, wherein the optical illumination source is a mercury lamp.

4. The apparatus of claim 1, wherein the probe has an absorption spectrum central wavelength related to a geometric property of the probe.

5. The apparatus of claim 4, wherein the geometric property is an aspect ratio of a rod.

6. The apparatus of claim 5, wherein the aspect ratio is between 1.0 and about 7 and the rod is made of gold.

7. The apparatus of claim 1, wherein the probe is functionalized by attaching a biomarker thereto.

8. The apparatus of claim 7, wherein the biomarker is an antigen.

9. The apparatus of claim 8, wherein the antigen is specific to a cell surface marker.

10. The apparatus of claim 9, wherein the cell surface marker is a protein.

11. The apparatus of claim 1, wherein the probe comprises a gold rod and an antigen attached thereto.

12. The apparatus of claim 11, wherein the probe is a plurality of probes having differing optical properties, and at least two probes have different antigens attached thereto.

13. The apparatus of claim 12, wherein a first probe of the plurality of probes has an antigen attached thereto corresponding to a surface marker that is substantially uniformly expressed in the cell population.

14. The apparatus of claim 13, wherein an abundance of cells having a second probe attached thereto is computed using a measured abundance of cells having the at least the first probe attached thereto as a reference quantity.

15. The apparatus of claim 14, wherein the antigen attached to the first probe is different from the antigen attached to the second probe.

16. The apparatus of claim 1, further comprising a camera disposed so as to receive an image from the optical system.

17. The apparatus of claim 1, wherein the functionalized probe includes a magnetic particle.

18. The apparatus of claim 1, wherein the optical spectrum analyzer is a hyperspectral imaging device.

19.-36. (canceled)

37. A computer program product, stored on a non-transient computer readable media, comprising:
instructions for configuring a computer to:
accept data from an optical spectrum analyzer the data including an optical radiation spectrum of a functionalized probe; and
determine a number of cells of a population of cells having one or more functionalized probes types attached thererto.

38. The computer program product of claim 37, further comprising:
computing the relative abundance of at least one immunophenotype with respect to the abundance of a reference immunophenotype.

39. The computer program product of claim 38, wherein the reference immunophenotype is expressed relatively uniformly throughout a cell population being analyzed.

40. The computer program product of claim 37, wherein the optical spectrum analyzer is a hyperspectral imaging device.

41.-44. (canceled)
